(19) United States
(12) Patent Application Publication
(10) Pub. No.: US 2009/0005419 A1
(43) Pub. Date: Jan. 1, 2009

(54) Organic Compounds

(76) Inventors: Manfred Auer, Moedling (AT); Nicole-Claudia Meisner, Wien (AT); Martin Hintersteiner, Wien (AT); Torsten Schindler, Wien (AT); Hubert Gstach, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/815,235

(22) PCT Filed: Mar. 1, 2006
(86) PCT No.: PCT/EP06/01866

§ 371 Date: Apr. 15, 2008

(30) Foreign Application Priority Data
Mar. 3, 2005 (GB) 0504428.4
Mar. 3, 2005 (GB) 0504430.0
Mar. 3, 2005 (GB) 0504431.8

(51) Int. Cl. A61K 31/454; G01N 33/566; C07D 401/02; A61K 31/4439; C07D 401/14

(52) U.S. Cl. 514/326; 436/501; 546/279.1; 514/343; 546/208

(57) ABSTRACT

The invention relates to an assay for identifying an agent that modulates the interaction of a mRNA with a target protein, e.g. HuR and to organic compounds of formula I

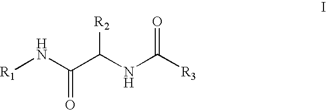

wherein R₁, R₂ and R₃ are as defined above and their use, e.g. as an inhibitor on the complex-formation of an ARE-containing mRNA and a target protein.

The present invention relates to organic compounds, and to an assay for identifying an agent that modulates the interaction of a mRNA with a target protein, e.g. ELAVL1 protein.

The expression of a vast number of genes is controlled at the level of messenger RNA. In particular, the rapid response of early response genes and, hence, various targets of disease relevance to a certain stimulus is often promoted by posttranscriptional control mechanisms. These processes are mainly mediated by regulatory proteins or factors acting on the messenger RNA. The inhibition or modulation of such regulatory target mRNA-protein interactions represents an attractive approach for therapeutical intervention.

To search for low molecular weight inhibitors of mRNA regulation, an assay to monitor the targeted mRNA-protein interaction, ideally in homogeneous solution is needed. The assay should further be suitable for implementation in a high throughput screening (HTS) environment, which is today most often based on fluorescence detection.

Direct binding of relatively small proteins, e.g. the mRNA stabilizing protein HuR (36 kD), to long mRNAs or -subfragments (281 to 1643 nucleotides, 100-500 kD) cannot be detected based on the relative increase in size that the fluorescently labeled RNA subsides upon complex formation. For this reason, spectroscopic methods which are based on rotational or translational diffusion time detection (such as e.g. Fluorescence Correlation Spectroscopy (FCS), 2D-FIDA-anisotropy or other fluorescence polarization measurements) can readily be excluded.

The assay of the present invention represents a novel method to monitor mRNA-protein interactions in homogeneous solution under true equilibrium conditions. It applies e.g. a highly sensitive single molecule detection and is therefore immediately adaptable to HTS platforms such as Evotec MarkII/III. Due to the high sensitivity and precision of the detection, e.g. the confocal detection, the assay of the present invention represents an attractive alternative to conventional electrophoretic mobility shift-, filter binding or nuclease protection assays. Also, an adaptation to conventional macroscopic fluorescence intensity detection methodologies (e.g. fluorescence plate readers) will be straightforward and does not necessarily require the availability of a confocal instrument.

In one aspect the present invention provides an assay for identifying an agent that modulates the interaction of a mRNA with a target protein comprising
a) providing a labeled mRNA having a length of at least 100 nucleotides, optionally as a homogenous solution, which label is a substance sensitive to changes in the surrounding of the mRNA,
b) contacting a target protein, e.g, HuR protein, with the mRNA provided in step a) in the absence and in the presence of a candidate compound which is expected to modulate the interaction of said mRNA with said target protein, e.g., HuR protein, for a sufficient period of time so that a complex between said mRNA and said target protein, e.g., HuR protein can be formed,
c) detecting the complex formed in step b),
d) determining whether there is a difference in the amount of complex formed in case a candidate compound was absent or present in step b), and
e) choosing a candidate compound where a difference is determined in step d) as an agent.

The assay principle may be described as follows (e.g. as given in FIG. 1): The mRNA is labeled, e.g. with Cy3 at the 3′ end by hydrazine aldehyde linkage chemistry. Upon binding of a target protein to the labeled mRNA, the quantum yield of the label, e.g. Cy3, changes, e.g. increases, and this change provides a readout for the interaction of mRNA with a target protein. This effect does apparently not involve direct contacts between the protein and the label and is reproducibly observed even for mRNAs with target protein, e.g. HuR, binding sites distant from the 3′ terminal label. We currently conclude that this may be caused by a change in the 3-dimensional RNA conformation upon complex formation with the target protein, translated through long range effects and attributed to the environment sensitivity of a label, e.g. Cy3 (see e.g. Mujumdar R. B. et al., Bioconj Chem. 1993, 4(2) 105-11). Reduced protein-mRNA complex formation attributed to a potential inhibitor will be detected as a decrease in the readout, e.g. total fluorescence intensity or a reduced particle number of species with higher molecular brightness.

In a preferred embodiment, a target protein is a HuR protein.

The mRNA may be an ARE-containing mRNA and includes e.g. inflammatory targets including AREs from TNF-α, IL-1β, IL-2, IL-8, Cox-2, IL-4 or AT-R1 but also to other ARE-regulated target families. For instance, proto-oncogenes like c-myc, c-jun or c-fos.

In another aspect of the present invention the mRNA is selected from the group consisting of the sequences encoding IL-2, IL-1β and TNF-α, or an ARE-containing fragments of such sequences.

In another aspect of the present invention the mRNA has a length between 100 and 500 nucleotides, preferably 300 nucleotides.

The label may be one as conventional, e.g. biotin or an enzyme such as alkaline phosphatase (AP), horse radish peroxidase (HRP) or peroxidase (POD) or a fluorescent molecule, e.g. a fluorescent dye. Preferably the label is a fluorescence dye, such as e.g. Cy3 or Cy5, e.g. Cy3.

In a preferred aspect of the present invention the label is a fluorescence label, e.g. Cy3 or Cy5.

The target protein may be any proteins known to bind to mRNA, wherein such binding cause changes in the 3-dimensional RNA conformation. In another aspect of the present invention the target protein is ELAVL1 (=HuR) which binds to ARE-containing mRNA.

For detecting the complex formed detection means may be used. Such detection means include those as conventional in the field of assays, such as e.g. fluorescence detection measurements. Detection means used in the present invention comprise molecules which recognize the labeled mRNA.

Using e.g. the 1-dimensional intensity dependent confocal fluorescence detection method FIDA (Fluorescence Intensity Distribution Analysis), the binding of a target protein, e.g. of HuR, to its target mRNAs can be followed directly and in homogeneous solution in a size independent way.

In another aspect of the present invention the complex is detected by measurement of the fluorescence intensity.

Optionally a complex formed can be separated from uncomplexed fractions. The separation can be carried out according, e.g. analogously, to methods as conventional, e.g. chromatographically, e.g. size exclusion chromatography.

A candidate compound includes compound(s)(libraries) from which its modulating effect on the interaction of a mRNA with a target protein can be determined. Compound (libraries) include for example oligopeptides, polypeptides, proteins, antibodies, mimetics, small molecules, e.g. low molecular weight compounds (LMW's).

An agent is a compound which influences (inhibits) the interaction of a mRNA with a target protein, e.g. detected, in step d) in an assay provided by the present invention.

An agent is one of the chosen candidate compounds and may include oligopeptides, polypeptides, proteins, antibodies, mimetics, small molecules, e.g. low molecular weight compounds (LMW's). An agent includes one or more agents, e.g. a combination of agents.

In another aspect the present invention provides an assay of the present invention for high throughput screening.

In another aspect the present invention provides a kit comprising
a labeled mRNA, e.g. fluorescence labeled,
a target protein,
instructions for using such a kit, and
optionally a candidate compound.

Such kit as provided by the present invention may further comprise a substantial component including an appropriate environment of a sample to be tested and, e.g. appropriate means to determine the effect of a candidate compound in a sample to be tested.

The present invention further relates to organic compounds identified by the screening assay described above.

In another aspect, the present invention provides a compound of formula

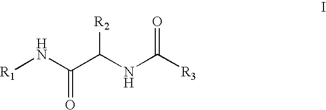

wherein

R₁ is (C₁₋₄)alkyl substituted by unsubstituted or substituted (C₆₋₁₈)aryl or heterocyclyl having or 6 ring members and 1 to 4 heteroatoms selected from the group consisting of N, O and S,

R₂ is (C₁₋₄)alkyl substituted by hydroxyl, carboxyl, amino or unsubstituted or substituted (C₆₋₁₈)aryl, or unsubstituted or substituted (C₆₋₁₈)aryl, and

R₃ is unsubstituted or substituted (C₆₋₁₈)aryl or heterocyclyl having 5 or 6 ring members and 1 to 4 heteroatoms selected from the group consisting of N, O and S.

In a preferred aspect in a compound of formula I, R₁ is (C₁₋₃)alkyl substituted by unsubstituted or substituted phenyl or heterocyclyl having 5 ring members and N as a heteroatom,

R₂ is (C₁₋₂)alkyl substituted by hydroxyl, carboxyl, amino or unsubstituted or substituted phenyl, or unsubstituted or substituted phenyl, and

R₃ is unsubstituted or substituted phenyl or heterocyclyl having 5 or 6 ring members and N as a heteroatom.

In another preferred aspect in a compound of formula I, R₁ is methyl substituted by p-methyl-phenyl or n-propyl substituted by 1-pyrrolidin-2-one, R₂ is methyl substituted by carboxyl, methyl substituted by p-methyl-phenyl, methyl substituted by 1H-indol-3-yl, ethyl substituted by hydroxyl, ethyl substituted by amino or p-methyl-phenyl, and

R₃ is a compound of formula

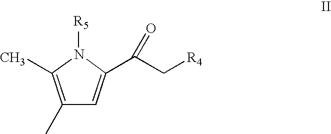

wherein
R₄ is 1-piperidine or 1-(p-aminocarbonyl)-piperidine,
R₅ is methoxyethyl, benzyl or (p-methoxy-phenyl)-ethyl, or
a compound of formula

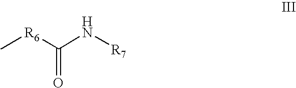

wherein
R₆ is p-phenyl or m-pyridine and
R₇ is methyl substituted by m-methoxy-phenyl or 1-aminocarbonyl-2-hydroxy-propyl.

If not otherwise defined herein alkyl includes (C₁₋₈)alkyl, e.g. (C₁₋₄)alkyl. Aryl includes (C₆₋₁₈)aryl, e.g. phenyl. Heterocyclyl includes a 5 or 6 membered ring having 1 to 4 heteroatoms selected from S, O and N; e.g. N; such as e.g. piperidine, pyridine and pyrrolidine, optionally anellated with a further ring (system), e.g. anellated with a phenyl ring; e.g. or anellated with a heterocyclyl ring. Alkyl, aryl and heterocyclyl include unsubstituted or substituted alkyl, aryl or heterocyclyl, e.g. substituted by groups which are conventional in organic chemistry. Amino includes unsubstituted and substituted amine, e.g. alkyl- and dialkylamine.

In a compound of formula I each single defined substituent may be a preferred substituent, e.g. independently of each other substituent defined.

In yet another aspect, the present invention provides a compound of formula I

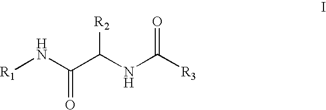

wherein
a) R₁ is n-propyl substituted by 1-pyrrolidin-2-one, R₂ is ethyl substituted by amino and R₃ is a compound of formula

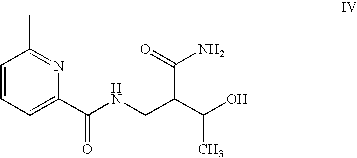

b) R₁ is methyl substituted by p-methyl-phenyl, R₂ is ethyl substituted by amino and R₃ is a compound of formula

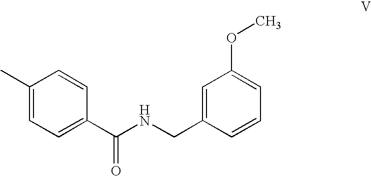

c) R₁ is methyl substituted by p-methyl-phenyl, R₂ is methyl substituted by p-aminomethyl-phenyl and R₃ is a compound as defined in b),
d) R₁ is n-propyl substituted by 1-pyrrolidin-2-one, R₂ is ethyl substituted by hydroxyl and R₃ is a compound of formula

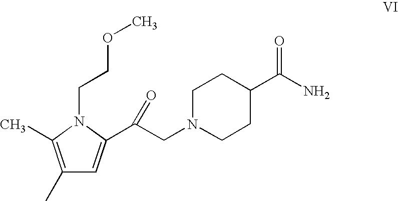

e) R₁ is n-propyl substituted by 1-pyrrolidin-2-one, R₂ is methyl substituted by carboxyl and R₃ is a compound of formula

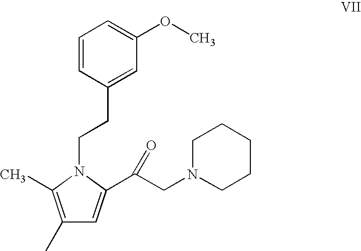

f) R₁ is n-propyl substituted by 1-pyrrolidin-2-one, R₂ is methyl substituted by 1H-indol-3-yl and R₃ is a compound of formula

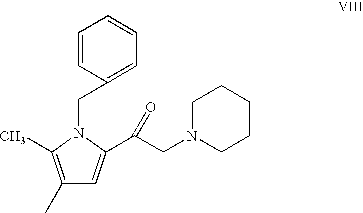

g) R₁ is n-propyl substituted by 1-pyrrolidin-2-one, R₂ is methyl substituted by p-methyl-phenyl and R₃ is a compound as defined in f).

Compounds provided by the present invention are hereinafter designated as “compound(s) of (according to) the present invention”. A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes. A salt of a compound of the present invention includes a metal salt or an acid addition salt. Metal salts include for example alkali or earth alkali salts; acid addition salts include salts of a compound of formula I with an acid, e.g. hydrogen fumaric acid, fumaric acid, naphthalin-1,5-sulphonic acid, hydrochloric acid, deuterochloric acid; preferably hydrochloric acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of pure isomers or mixtures thereof; e.g. optical isomers, diastereoisomers, cis/trans isomers. A compound of the present invention may e.g. contain asymmetric carbon atoms and may thus exist in the form of enantiomers or diastereoisomers and mixtures thereof, e.g. racemates. Any asymmetric carbon atom may be present in the (R)-, (S)- or (R,S)-configuration, preferably in the (R)- or (S)-configuration.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

In another aspect the present invention provides a process for the production of a compound of formula I, wherein
—R₃ is a compound of formula III comprising the steps

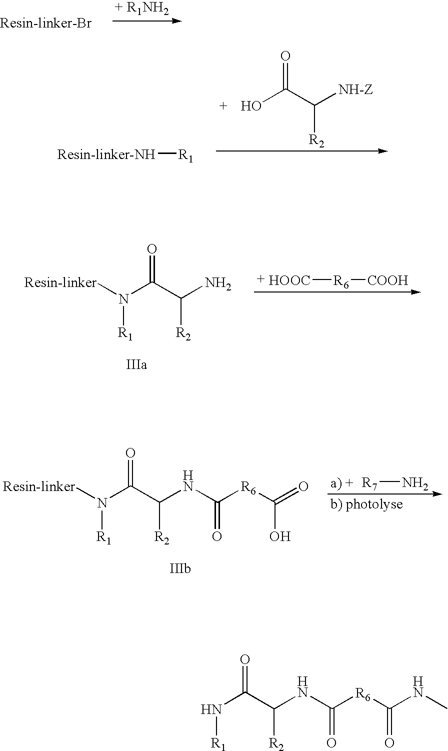

wherein R₁, R₂, R₆ and R₇ are as defined above to obtain a compound of formula I, and isolating a compound of formula I obtained from the reaction mixture OR
R₃ is a compound of formula II comprising the steps

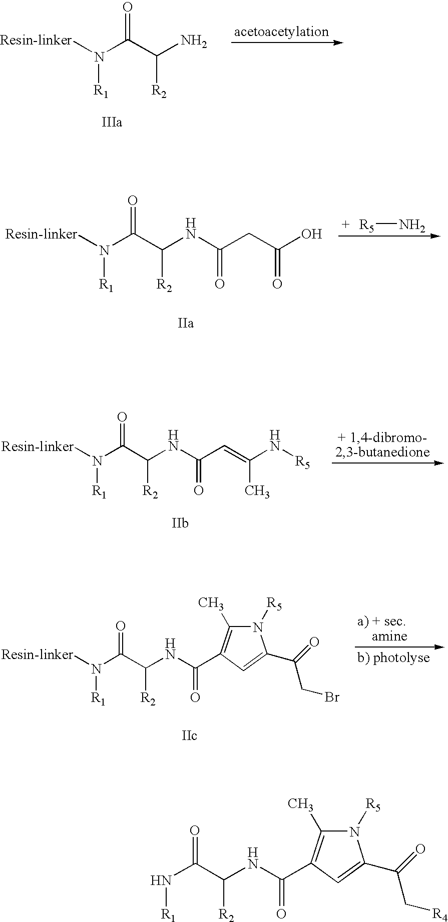

wherein R₁, R₂, R₄ and R₅ are as defined above to obtain a compound of formula I, and isolating a compound of formula I obtained from the reaction mixture.

In an intermediate of formula IIa, IIIb, IIa, IIb or IIc (starting materials), functional groups, if present, optionally may be in protected form or in the form of a salt, if a salt-forming group is present. Protecting groups, optionally present, may be removed at an appropriate stage, e.g. according, e.g. analogously, to a method as conventional.

A compound of formula I thus obtained may be converted into another compound of formula I, e.g. or a compound of formula I obtained in free form may be converted into a salt of a compound of formula I and vice versa.

Intermediates (starting materials) of formula IIIa, IIIb, IIa, IIb or IIc are known or may be prepared according, e.g. analogously, to a method as conventional or as described herein.

Any compound described herein, e.g. a compound of the present invention and intermediates of formula IIIa, IIIb, IIIa, IIb or lic may be prepared as appropriate, e.g. according, e.g. analogously, to a method as conventional, e.g. or as specified herein.

In another aspect, the present invention provides the use of a compound of the present invention as an inhibitor of the complex-formation of an ARE-containing mRNA and a target protein, e.g., a HuR protein.

In a preferred aspect, the ARE-containing mRNA is selected from the group consisting of IL-2, IL-1β and TNF-α.

The compounds of the present invention, e.g., including a compound of formula I, exhibit pharmacological activity and are therefore useful as pharmaceuticals.

In another aspect the present invention provides a compound of the present invention for use as a pharmaceutical.

For pharmaceutical use a compound of the present invention includes one or more, preferably one, compounds of the present invention, e.g. a combination of two or more compounds of the present invention.

In another aspect the present invention provides a pharmaceutical composition comprising a compound of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such compositions may be manufactured according, e.g. analogously to a method as conventional, e.g. by mixing, granulating, coating, dissolving or lyophilizing processes. Unit dosage forms may contain, for example, from about 0.5 mg to about 1000 mg, such as 1 mg to about 500 mg.

In another aspect the present invention provides the use of a compound of the present invention for the manufacture of a medicament, e.g. a pharmaceutical composition, for the treatment of a disorder having an etiology associated with the production of a substance selected from the group consisting of cytokine, growth factor, proto-oncogene or a viral protein, preferably the agent is selected from the group consisting of IL-1, IL-2, IL-3, IL-4, IL-8, GM-CSF, TNF-α, VEGF, AT-R1, Cox-2, c-fos and c-myc.

In a further aspect the present invention provides a method of treatment of a disorder having an etiology associated with the production of a substance selected from the group consisting of cytokine, growth factor, proto-oncogene or a viral protein, preferably the agent is selected from the group consisting of IL-1, IL-2, IL-3, IL-4, IL-8, GM-CSF, TNF-α, VEGF, AT-R1, Cox-2, c-fos and c-myc, which treatment comprises administering to a subject in need of such treatment an effective amount of a compound of the present invention; e.g. in the form of a pharmaceutical composition.

Treatment includes treatment and prophylaxis.

For such treatment, the appropriate dosage will, of course, vary depending upon, for example, the chemical nature and the pharmacokinetic data of a compound of the present invention employed, the individual host, the mode of administration and the nature and severity of the conditions being treated. However, in general, for satisfactory results in larger mammals, for example humans, an indicated daily dosage is in the range from about 0.01 g to about 1.0 g, of a compound of the present invention; conveniently administered, for example, in divided doses up to four times a day.

A compound of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration;

e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

The compounds of the present invention may be administered in the form of a pharmaceutically acceptable salt, e.g. an acid addition salt or metal salt; or in free form; optionally in the form of a solvate. The compounds of the present invention in the form of a salt exhibit the same order of activity as the compounds of the present invention in free form; optionally in the form of a solvate.

A compound of the present invention may be used for pharmaceutical treatment according to the present invention alone or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

### DESCRIPTION OF THE FIGURES

FIG. 1: Assay principle (schematically)

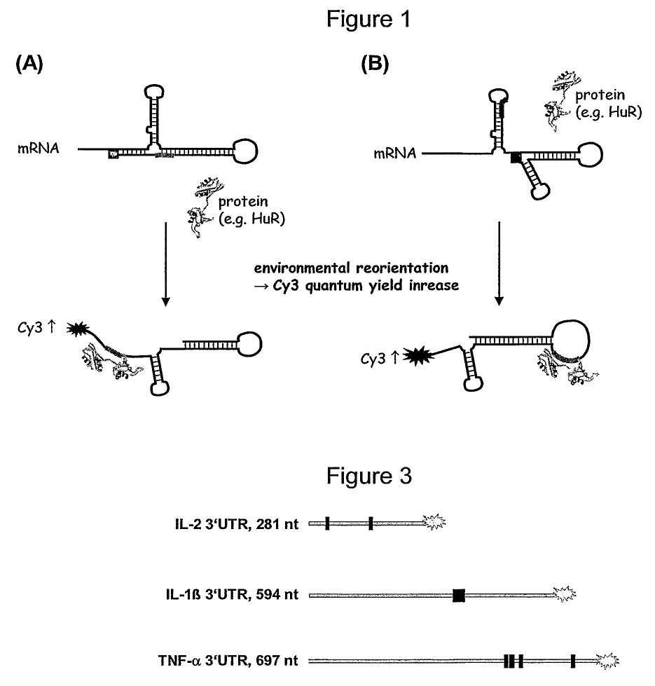

Upon protein binding to the labelled, e.g. Cy3-labeled, mRNA (here: 3′-terminal label), the quantum yield of the environment sensitive label, e.g. Cy3, increases. This increase is detectable as an increase in an appropriate readout, such as e.g. the molecular brightness by the FIDA algorithm or e.g. as total fluorescence intensity increase using an ensemble averaging readout. This effect is equally observed for protein bindings sites proximal (A) or distant (B) to the label, e.g. the Cy3 label, within the mRNA sequence.

FIG. 2: Exemplary Cy3 FIDA assay mRNA-protein binding curves

Exemplary binding curves for the interaction of HuR_{fl} with the 3′ terminally Cy3 labeled 3′UTRs (=untranslated regions) of IL-2 (A), IL-1β (B) and TNF-α (C) are shown. Dissociation constants (Kd) are determined according to Eq.1 as given below in the Examples. To exclude nonspecific HuR interaction with Cy3, a negative control is performed with the free dye (D). The Cy3 molecular brightness remains also unchanged upon titration of the IL-2 3′UTR with BSA (=bovine serum albumin) as control protein (E). The concentration of 3′terminally Cy3 labeled RNA is 0.5 nM in all experiments. 5S RNA, which does not contain any HuR binding site, is present at 100 nM in all samples as nonspecific competitor RNA. However, almost identical binding curves are recorded in absence of any competitor RNA (shown for the TNF-α 3′UTR, (F)).

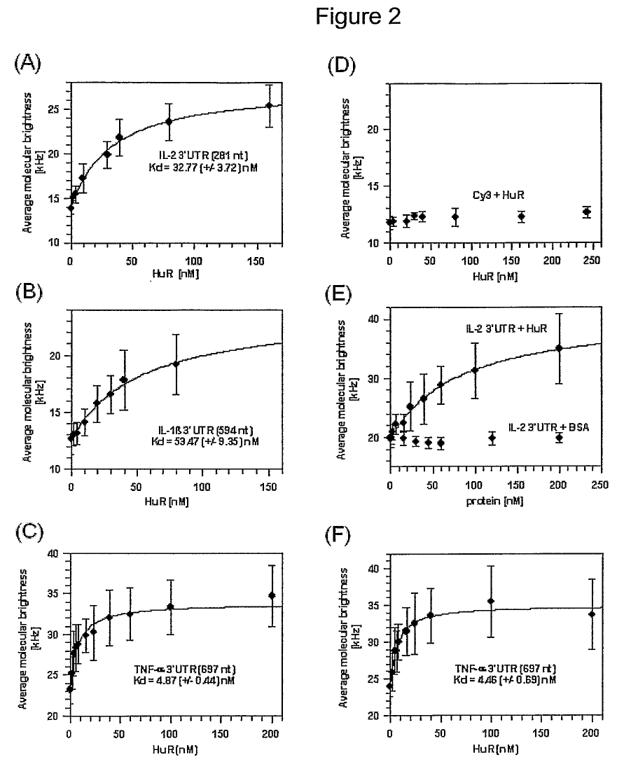

FIG. 3: RNA transcripts for binding experiments shown in FIG. 2

In-vitro transcripts of the 3′UTRs of human IL-2, IL-1β and TNF-α (GenBank accession numbers NM_{—}000589, NM_{—}000576 and NM_{—}000594; positions 707-1035, 897-1490 and 872-1568, respectively) are 3′ terminally labeled with Cy3. Although the HuR binding sites (shown in blue) are of different (sequence) proximity to the 3′terminal label, the Cy3 quantum yield increase upon protein binding is observed consistently.

In the following examples all temperatures are in degree centigrade and are uncorrected.

The following ABBREVIATIONS are used:

BSA bovine serum albumin
Cy3 fluorescence dye
FCS Fluorescence correlation spectroscopy
FIDA Fluorescence intensity distribution analysis
HuR_{fl} full length HuR protein
HuR_{1,2} shortened variant of HuR_{fl}

### OD Optical Density

### PBS Phosphate Buffered Saline

### RP-HPLC Reverse Phase High Performance Liquid

### Chromatography

rt room temperature
CONA confocal nanoscanning
DMF dimethylformamide
HuR₁₂ a shortened variant of HuR
TMR 5′carboxytetramethylrhodamine
rt room temperature

### EXAMPLES

### A/Screening Assay

In vitro transcribed mRNAs are 3′terminally labeled with hydrazide-activated Cy3 (Amersham Biosciences) following standard protocols (e.g. Qin P Z et al., Methods 1999; 18(1):60-70). The labeled RNA is purified by RP-HPLC. A 1:1 stoichiometry is controlled by UV/VIS spectroscopy. The Cy3-labeled mRNA is thermally denatured for 2 minutes at 800 in assay buffer (PBS, 0.1% (w/v) Pluronic F-127, 5 mM MgCl₂) and refolded by cooling to rt at a gradient of −0.13° s⁻¹. The final concentration of labeled RNA in each sample is 0.5 nM, which ensures an average of <1 fluorescent particles in the contocal volume in the setup described below. The accurate concentration in each sample is determined based on the particle number derived from a parallel FCS evaluation and the size of the confocal volume, as given by the adjustment parameters for the point spread function. Fluorescently labeled RNA is titrated against increasing concentrations of recombinant HuR_{fl} or HuR_{1,2}. HuR-mRNA complex formation is monitored under true equilibrium conditions by determination of the molecular brightness with 1 D-FIDA (e.g. Kask P. et al., Introduction to the theory of fluorescence intensity distribution analysis. FLUORESCENCE CORRELATION SPECTROSCOPY: THEORY AND APPLICATIONS 65 PG. 396-409. 2001 [Figures]-409; Kask P. et al., Fluorescence-intensity distribution analysis and its application in biomolecular detection technology. Proceedings of the National Academy of Sciences of the United States of America 96[24], 13756-13761. 23-11-1999). Alternatively, the Cy3 fluorescence intensity can be measured using conventional ensemble averaging detection methods, e.g. fluorescence plate readers.

FIDA measurements are performed in 96- or 384-well glass bottom microtiter plates (Whatman) on an EvotecOAl PickoScreen 3 instrument at ambient temperature (constant at 23.5°). The Olympus inverted microscope IX70 based instrument is equipped with 2 fluorescence detectors, a dichroic mirror in the fluorescence excitation path. A HeNe laser (λ=543 nm, laser power=478 pW) is used for fluorescence excitation. The excitation laser light is blocked from the optical detection path by an interference barrier filter with OD=5. Cy3 or TMR in assay buffer (c=0.5 nM) is used for the adjustment of the confocal pinhole (70 μm). The 1D-FIDA signal is averaged from 20 consecutive measurements (10 seconds each). Analysis of the raw data with the FIDA algorithm follows to extract

(a) the average molecular brightness equivalent to a conventional ensemble averaging measurement or
(b) the equilibrium concentrations of the individual components (free mRNA versus complex) with their respective molecular brightnesses.

The molecular brightness data are fitted by nonlinear least square regression (GraFit 5.0.3, Erithacus software, London) to extract the equilibrium dissociation constant K_{d}, using the exact algebraic solution of the binding equation derived from the law of mass action describing

(a) the average steady-state signal q in dependence of degree of (1:1) complex formation as determined by the dissociation constant Kd

$$q = {q}_{min} + \frac{( {q}_{max} - {q}_{min} ) * ⌊ \begin{matrix}( [ {mRNA}_{0} ] + [ {HuR}_{0} ] + {Kd}_{app} ) - \\ \sqrt{\begin{matrix}{( [ {mRNA}_{0} ] + [ {HuR}_{0} ] + {Kd}_{app} )}^{2} - \\ 4 * [ {mRNA}_{0} ] * [ {HuR}_{0} ]\end{matrix}}\end{matrix} ⌋}{2 * [ {mRNA}_{0} ]} \qquad Eq . 1$$

where [mRNA₀]: total concentration of RNA, [HuR₀]: total concentration of HuR, qₘᵢₙ: molecular brightness of free RNA, qₘₐₓ: molecular brightness of RNA-HuR complex, q: average molecular brightness for the steady-state equilibrium at the given HuRo and RNAo concentrations;
(b) equilibrium concentrations of free and protein bound mRNA ([mRNA_{free}] and [mRNA HuR], respectively, directly delivered by the FIDA analysis) as determined by the dissociation constant K_{d}

$${K}_{d} = \frac{⌊ {mRNA}_{free} ⌋ \times ( [ {HuR}_{0} ] - [ mRNA \cdot HuR ] )}{[ mRNA \cdot HuR ]} \qquad Eq . 2$$

Exemplary binding curves for interaction between HuR and individual Cy3 labeled target mRNAs are depicted in FIG. 2 (all presented data are averages of 20 individual FIDA measurements and representative for at least three independent experiments).

To resume, the present assay provides a novel approach to monitor (regulatory) mRNA protein interactions in homogeneous solution. The assay combines the advantages of true equilibrium conditions with high detection sensitivity and precision and is suitable to screen for potential inhibitors or modulators of the interaction in a high throughput screen. With the vast number of posttranscriptionally regulated disease relevant targets, the described assay will serve as a basis for therapeutic intervention in cancer, inflammatory, viral, or allergic disease, based on a novel, RNA-targeting approach.

### B) Analytical Methods

a) HPLC: For analytical separations an Abimed (D-Langenfeld) HPLC system is used consisting of 2 pump units 306, a dynamic mixing chamber module 811C, a manometric pressure module 805, an UV-Detector UV/VIS 155 and an autoinjector 234. The separation is performed on an analytical column GromSil 500DS-5 ST (3 μm, 120×2 mm) manufactured by Grom (D-Herrenberg). A gradient of H₂O/0.1% TFA (v/v) (eluent A) and acetonitrile/0.1% TFA (v/v) (eluent B) with a flow rate of 0.4 mL/minutes is used. UV-traces are measured at λ=214 and λ=305 nm, respectively. The purity of the products is assigned on the basis of the peak areas determined at λ=214 nm.
b) ES-MS: ES-MS-analysis is performed on a Micromass (Altrinchan/UK) Quattro II triple quadrupole mass spectrometer with a Waters (D-Eschborn) 515 make-up pump (isocratic flow of 60 μL/min acetonitrile/water 1:1, containing 0.1% formic acid) and a Abimed (D-Langenfeld) Gilson 232× autosampler
c) LC-MS: LC-MS-analysis is performed on a Waters-Micromass (D-Eschborn) ZQ mass spectrometer with a HPLC-system 2790 Alliance HT separation module and a 996 Diode Array Detector. An analytical column GromSil120 ODS-5 ST (3 μm, 60×2 mm) manufactured by Grom (D-Herrenberg) is used. A gradient of H₂O/0.1% TFA (v/v) (eluent A) and acetonitrile/0.1% TFA (v/v) (eluent B) with a flow rate of 0.6 mL/minutes is used.
d) Preparative HPLC: For preparative separations an Abimed (D-Langenfeld) HPLC system 1014 is used consisting of a pump unit 322, an UV-Detector UV/VIS 151 (□=305 nm) and a Gilson 215 Liquid-Handler. The separation is performed on a preparative column Purospher RP18 (5 μm, 125×25 mm) manufactured by Merck (D-Darmstadt). A gradient of H₂O/0.1% TFA (v/v) (eluent A) and acetonitrile/0.1% TFA (v/v) (eluent B) with a flow rate of 30 mL/minutes is used.

### Examples 1 to 3

### Synthesis of Compounds 1 to 3 are Carried Out According to SCHEME 1

### Scheme 1

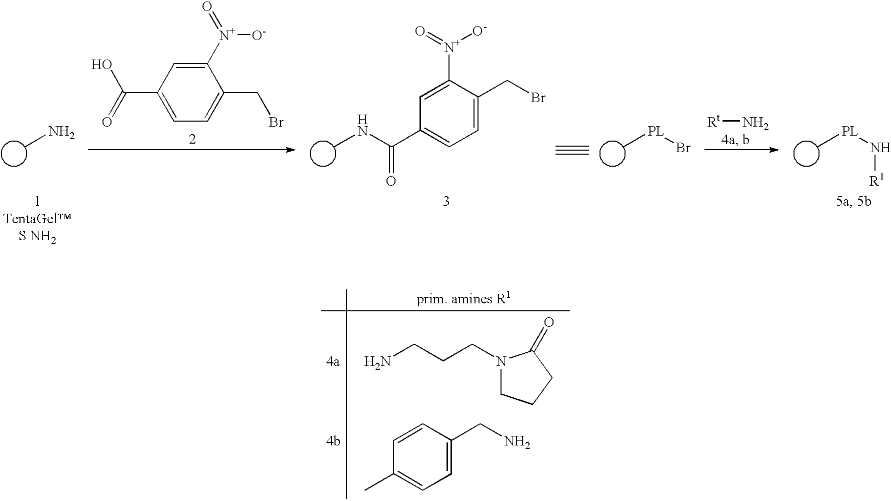

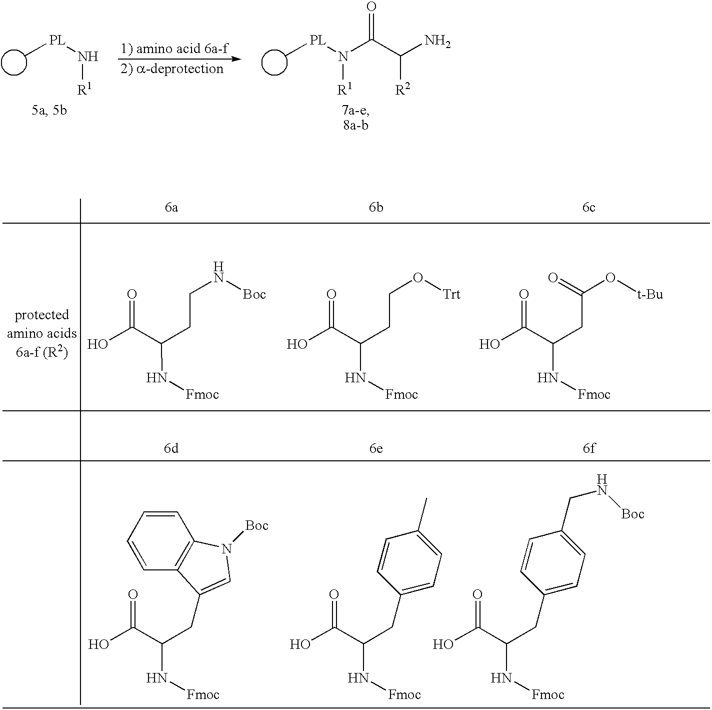

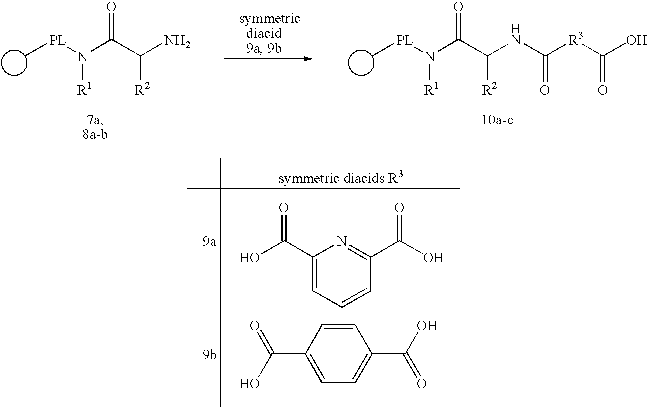

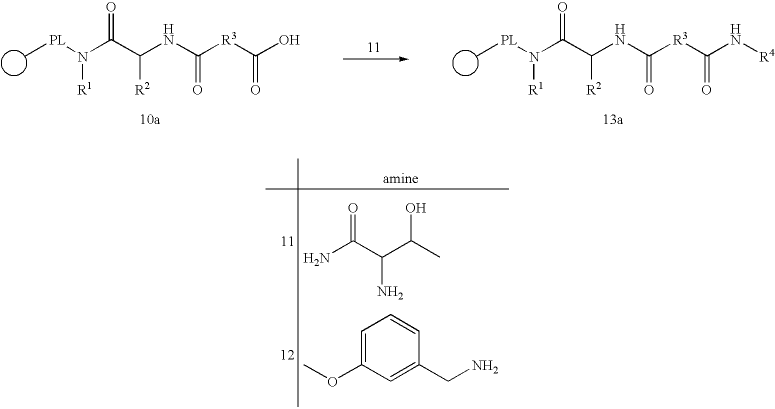

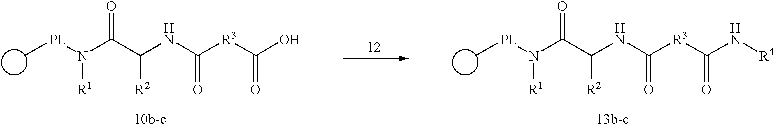

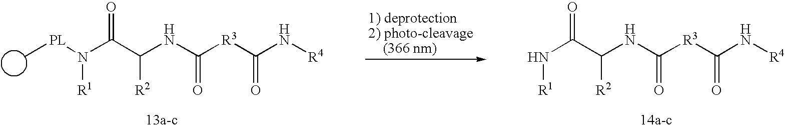

a) Loading of solid support with linker

4.2 g of TentaGel™ S NH₂-resin 1 (loading capacity =0.25 mmol/g) are pre-washed with DMF (4×20 mL). 0.82 g of 4-Bromomethyl-3-nitrobenzoic acid 2 and 0.482 g of HOBt*H₂O are dissolved in 60 mL of 8% DMF in DCM (v/v). 487 μL of DIC are added. After stirring at rt for 30 minutes the solution obtained is transferred to the pre-washed TentaGel™ S NH₂-resin 1. The mixture obtained is shaken at rt for 19 hours. The resin 3 obtained is filtered off, washed with DMF (6×50 mL), DCM (6×50 mL) and MeOH (6×50 mL) and dried under reduced pressure. Completion of the reaction is verified by a negative Kaiser-ninhydrin test.

b) Reactions of resin 3 with primary amines 4a, 4b

To 3.18 g of resin 3 in 20 mL of DMSO 2.133 g of 1-(3-aminopropyl)-pyrrolidin-2-one (4a) are added. To 1.27 g of resin 3 in 10 mL of DMSO 0.73 g of 4-methylbenzylamine (4b) are added. The mixtures obtained is shaken for 3 hours at rt. The resins 5a and 5b obtained are filtered off, washed with DMSO (3×6 mL), DMF (6×6 mL), DCM (6×6 mL), MeOH (6×6 mL) and dried under reduced pressure. The chloranil tests are positive.

c) Coupling of protected amino acids 6a-f onto resins 5a-b and cleavage of the N(α) protecting group (Fmoc)

0.65 g of resin 5a are divided into 5 aliquots. 0.65 g of resin 5b are divided into two equal portions. 0.45 mmol of each N-protected amino acids 6a-f (6a: N(α)-Fmoc-N(γ)-Boc-L-2,4-diaminobutyric acid, twice; 6b: Fmoc-(OTrt)-L-homoserine; 6c: Fmoc-Asp(OtBu)-OH; 6d: Fmoc-Trp(Boc)-OH; 6e: Fmoc-L-4-MePhe-OH; 6f: Fmoc-L-(4-Boc-aminomethyl)Phe-OH each are dissolved in solutions of 5 mL of DMF, 93 μL of DIC and 92 mg of HOBt*H₂O. The solutions obtained are stirred for 30 minutes at rt. The solution of activated 6a obtained is divided into 2 equal portions. The portions are transferred to an aliquot of resin 5a and resin 5b to give the side chain protected resins 7a and 8a, respectively. The solutions of activated 6b-e obtained are transferred to the remaining aliquots of resin 5a to give protected resins 7b-e. The solution containing 6f is transferred to the second aliquot of resin 5b to give protected resin 8b. After shaking for 16.5 hours at rt the resins obtained are filtered off and washed with DMF (9×10 mL), DCM (6×10 mL) and MeOH (6×10 mL). The fully protected resins 7a-e and 8a-b obtained are dried under reduced pressure. All chloranil tests are negative.

An aliquot (5 mL) of a stock solution of piperidine in DMF (400 mL, 1/1, v/v) is added to each of 0.66 g of the protected resins 7a-e and 8a-b for cleavage of the N(α)-Fmoc protecting group. The mixtures obtained are shaken for 30 minutes. The de-protected resins 7a-e, 8a-b obtained are filtered off and subjected to repeated washes with DMF (9×25 mL), DCM (6×25 mL) and MeOH (6×25 mL). The resins obtained are dried under reduced pressure. The Kaiser-ninhydrin tests are positive.

d) Synthesis of compounds 14a-c containing symmetric dicarboxylic acid substructures

Coupling of symmetric dicarboxylic acids 9a and 9b onto Fmoc-deprotected resins 7a and 8a-b

The symmetric dicarboxylic acid 9 (9a: pyridine-2,6-dicarboxylic acid; 9b: terephthalic acid, twice; 1.5 mmol, each) is dissolved in a solution of 115 mg of HOBt*H₂O, 485 mg of DIEA and 95 mg of DIC in 5 mL of DMF. The solution of 9a obtained is added to resin 7a to give resin 10a. The two aliquot solutions of 9b obtained are added to resins 8a and 8b to give resins 10b and 10c, respectively. After shaking for 20 hours at rt the resins 10a-c obtained are filtered off, washed with DMF (9×20 mL), DCM (6×10 mL), diethylether (6×10 mL), and dried under reduced pressure. The Kaiser-ninhydrin tests of all resin samples are negative.

e) Coupling of resin bound dicarboxylic acid mono-amides 10a-c with amino acid amide 11 and primary amine 12

A solution of 339 mg of pentafluorophenyl trifluoroacetate and 242 μL of pyridine in 5 mL of NMP is added to each of 650 mg of the resins 10a-c. The mixtures obtained are shaken for 2.5 hours at rt. The resins obtained are filtered off and washed with NMP (10×5 mL). 298 mg of (O-Trt)-protected L-threonine amide hydrochloride (11) are dissolved in a solution of 20.3 mg of anhydrous HOBt and 3 mmol of DIEA in 3.0 mL of NMP and added to 650 mg of the pre-activated resin 10a to give resin 13a. 182 mg of 3-Methoxybenzylamine (12) are dissolved in a solution of 40.6 mg of anhydrous HOBt and 1.5 mmol of DIEA) in 6.0 mg of NMP. The solution obtained is divided in 2 aliquots, which are added to the pre-activated resins 10b and 10c to give resins 13b and 13c, respectively. The mixtures obtained are shaken for 14.5 hours at rt. The resins obtained are filtered off, washed with NMP (6×10 mL), DMF (6×10 mL), DCM (6×10 mL), and MeOH (6×10 mL) and dried under reduced pressure.

f) Cleavage of the Boc-protecting group from side chains of amino acids 6

5 mL of a solution of 50% TFA in DCM (v/v) are added to 650 mg of each of the resins 13a-c. The mixtures obtained are shaken for 2 hours at rt. The resins obtained are filtered off, washed with 20% TFA in DCM (v/v, 3×5 mL), DMF (6×10 mL), DCM (6×10 mL) and EtOH abs (6×10 mL) and dried under reduced pressure.

g) Cleavage of the final compounds 14a-c from the resins 13a-c

5 mL of MeOH+1% TFA v/v) are added to each of the deprotected resins 13a-c. The resin containing glass vials are placed in a plastic carrier for photolysis. Photolysis is carried out at 366 nm for 90 minutes under stirring in a Stratagene, UV Strataliner™ 1800 equipped with a NEC Blacklight T5, FL8BI, 8W-lamp. The chamber is cooled during the photo-cleavage with a stream of compressed air. The resin materials obtained are filtered off and washed with DCM (2×5 mL). Solvent from the filtrates obtained is evaporated. The crude materials can further be subjected to purification by preparative HPLC.

### Example 1

### Pyridine-2,6-dicarboxylic acid 2-({3-amino-1-[3-(2-oxo-pyrrolidin-1-yl)-propylcarbamoyl]-propyl}-amide) 6-[(1-carbamoyl-2-hydroxy-propyl)-amide] is obtained

| | | |
|---|---|---|
| MW = 491.5552 | retention time: 3.8 minutes | LC-MS [M⁺H]⁺: 492 |

### Example 2

### N-[3-Amino-1-(4-methyl-benzylcarbamoyl)-propyl]-N′-(3-methoxy-benzyl)-terephthalamide is obtained

| | | |
|---|---|---|
| MW = 488.592 | retention time: 8.29 minutes | LC-MS [M⁺H]⁺: 489 |

### Example 3

### N-[2-(4-Aminomethyl-phenyl)-1-(4-methyl-benzylcarbamoyl)-ethyl]-N′-(3-methoxy-benzyl)-terephthalamide is Obtained

| | | |
|---|---|---|
| MW = 564.69 | retention time: 8.46 minutes | LC-MS [M⁺H]⁺: 565 |

### Examples 4 to 7

### Synthesis of Compounds 4 to 7 are Carried Out According to SCHEME 2

### Scheme 2

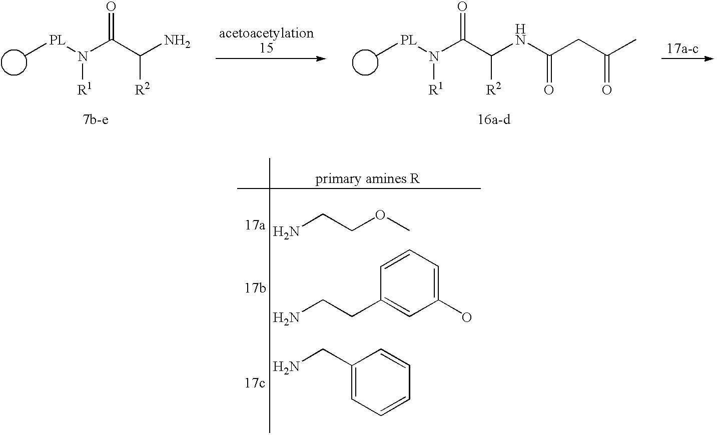

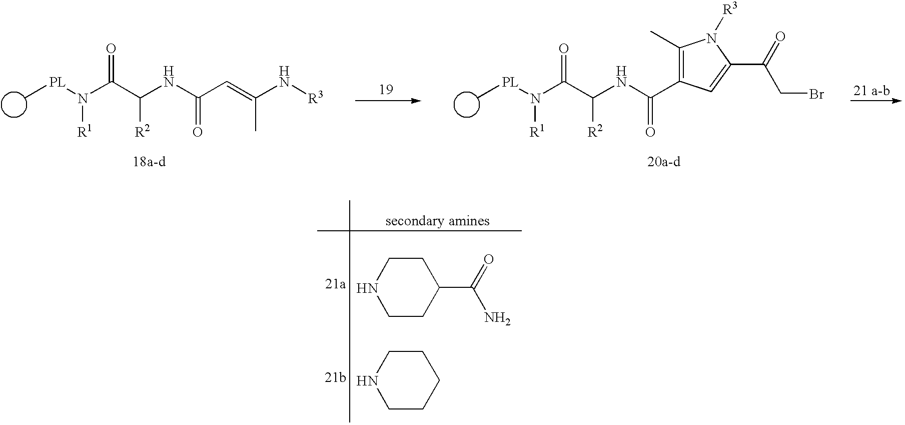

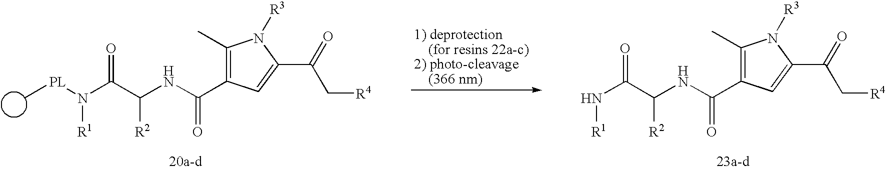

a) Acetoacetylation of resins 7b-e

N(α)-deprotected resins 7b-e are placed in 5 mL syringes. 359 mg of N-Hydroxysuccinimidyl acetoacetate 15 are dissolved in 17.0 mL of DCM. 310 mg of DIEA are added and aliquots of 4 mL of the stock solution are transferred to each syringe of 650 mg of each of the resins 7b-e. After shaking for 5 hours at rt the acetoacetylated resins 16a-d obtained are filtered off, washed with DCM (3×5 mL), DMF (6×5 mL), DCM (6×5 mL) and MeOH (6×5 mL) and dried. The Kaiser-ninhydrin tests are negative.

b) Reaction of resins 16a-d with primary amines 17a-c (enaminone formation) 5 mL of a stock solution of THF and TMOF (1/1, v/v) are added to 650 mg of each of the resins 16a-d. 1.5 mmol of each of the primary amines 17a-c (17a: 2-methoxyethylamine for reaction with resin 16a; 17b: 2-(3-methoxyphenyl)-ethylamine for reaction with resin 16b; twice 17c: benzylamine for reactions with resins 16c and 16d, respectively:) are added and the mixtures obtained are shaken for 18 hours at rt. The resins 18a-d obtained are filtered off, washed with THF/TMOF (1/1, v/v) (3×6 mL), DMF (6×6 mL), DCM (6×6 mL) and MeOH (6×6 mL) and dried.
c) Pyrrole synthesis from resin bound enaminones 18a-d with 1,4-dibromo-2,3-butanedione

230 mg of 2,6-di-tert.-Butylpyridine and 292 mg of 1,4-dibromo-2,3-butanedione 19 are dissolved in 24 mL of dioxane to give a stock solution. Aliquots of the stock solution obtained (6 mL/resin) are added to 650 mg of each of the resins 18a-d. The mixtures obtained are shaken for 1.5 hours at rt. The resins 20a-d obtained are filtered off, washed with dioxane (6×6 mL), DMF (6×6 mL), THF (6×6 mL) and DCM (6×6 mL).

d) Substitution of resin bound bromoacetyl-pyrroles 20a-d with secondary amines 21a and 21b

192 mg of each of the secondary amines 21 (21a: piperidine-4-carboxamide for reaction with 128 mg of resin 20a, 21b: piperidine for reaction with resins 128 g of 20b-d) and DMSO (4×6 mL) are added to 650 mg of each of the resins 20a-d. The mixtures obtained are shaken for 15.5 hours at rt. The resins 22a-d obtained are filtered off, washed with DMSO (3×1 mL), DMF (6×1 mL), DCM (6×1 mL) and MeOH (6×1 mL) and dried.

e) Cleavage of the protecting groups from side chains of amino acids 6 (resins 22a-c) 5 mL of a solution of 20% TFA in DCM (v/v) is added to each of the resins 22a-c to remove side chain protecting groups on R² (Trt, t-Bu, Boc). The mixtures obtained are shaken for 1 hour at rt. The deprotected resins 22a-c obtained are filtered off, washed with 20% TFA in DCM (v/v, 3×1 mL), DMF (6×2 mL), DCM (6×2 mL), EtOH (6×2 mL) and ether (3×2 mL) and dried.
f) Cleavage of the final compounds 23a-d from the resins 22a-d

5 mL; of acidic methanol and 1% v/v TFA are added to each of the deprotected resins 22a-c and non-TFA treated resin 22d. The glass vials are placed in a plastic carrier. Photolysis is carried out under stirring and irradiation at 366 nm for 90 miutes in a Stratagene® UV Strataliner™ 1800 equipped with a NEC Blacklight T5, FL8BI, 8W-lamp. The chamber is cooled during the photo-cleavage with a stream of compressed air. The resin materials obtained are filtered off and washed with DCM (2×5 mL). Solvent of the combined filtrates obtained is evaporated. The crude materials of the compounds may be further subjected to purification by preparative HPLC.

### Example 4

### 1-{2-[4-{3-Hydroxy-1-[3-(2-oxo-pyrrolidin-1-yl)-propylcarbamoyl]-propylcarbamoyl}-1-(2-methoxy-ethyl)-5-methyl-1H-pyrrol-2-yl]-2-oxo-ethyl}-piperidine-4-carboxylic acid amide is Obtained

| | | |
|---|---|---|
| MW = 576.699 | retention time: 2.24 minutes | LC-MS [M⁺H]⁺: 577 |

### Example 5

### 3-{[1-[2-(3-Methoxy-phenyl)-ethyl]-2-methyl-5-(2-piperidin-1-yl-acetyl)-1H-pyrrole-3-carbonyl]-amino}-N-[3-(2-oxo-pyrrolidin-1-yl)-propyl]-succinamic acid is Obtained

| | | |
|---|---|---|
| MW = 623.756 | retention time: 7.59 minutes | LC-MS [M⁺H]⁺: 624 |

### Example 6

### 1-Benzyl-2-methyl-5-(2-piperidin-1-yl-acetyl)-1H-pyrrole-3-carboxylic acid {2-(1H-indol-3-yl)-1-[3-(2-oxo-pyrrolidin-1-yl)-propylcarbamoyl]-ethyl}-amide is Obtained

| | | |
|---|---|---|
| MW = 650.828 | retention time: 8.248 minutes | LC-MS [M⁺H]⁺: 651 |

### Example 7

### 1-Benzyl-2-methyl-5-(2-piperidin-1-yl-acetyl)-1H-pyrrole-3-carboxylic acid {1-[3-(2-oxo-pyrrolidin-1-yl)-propylcarbamoyl]-2-p-tolyl-ethyl}-amide is Obtained

| | | |
|---|---|---|
| MW = 625.818 | retention time: 8.44 minutes | LC-MS [M⁺H]⁺: 626 |

### Example 8

### Identification of Small Molecular Inhibitors of HuR-Associated mRNA Stability Regulation

To identify small molecular inhibitors of HuR-associated mRNA stability regulation, 3 different complementing HT screens are carried out:

(1) A cellular reporter gene assay with firefly luciferase under control of the IL-2 or TNF-α AU-rich
element. This assay is designed to identify cellularly active, non-toxic inhibitors of ARE-mediated mRNA stabilization. The confirmed hits are tested in a control assay with the luciferase CDS under the IL-2 or TNF-α promoter to exclude compounds acting at the transcriptional level. By definition, the identified hit compounds interfere with posttranscriptional stabilization of short-lived ARE mRNAs at some level along the ARE pathway.
(2) In vitro screen for HuR-ARE inhibitors using confocal fluctuation spectroscopy. This assay uses 2D-FIDA to monitor binding of HuR₁₂, a shortened variant of HuR to a TMR labeled ARE RNA in solution. Compounds identified in this screen are supposed to interfere with HuR-ARE recognition by binding either to the ARE or to HuR₁₂.
(3) CONA with HuR. Combinatorial on-bead libraries are screened for high affinity HuR binders with CONA. After picking of hit beads and cleavage of on-bead binders from the solid support, the compound structures are decoded by PHPLC-MS. Binding of resynthesized hit compounds to HuR is tested in solution. As the primary result, the identified compounds represent high affinity HuR binders. Functionally, these may be inhibitors of any HuR activity including ARE recognition, nucleocytoplasmic shuttling and protection of the mRNA from ARE-dependent degradation.

The RNA fraction bound y is calculated from the anisotropy data based on y=(r−rₘᵢₙ)/((r−rₘᵢₙ)+Q(rₘₐₓ−r)) with r≡ measured anisotropy, rₘᵢₙ, rₘₐₓ≡ anisotropy of the free and HuR bound RNA, respectively, Q ≡ quenching. Dissociation constants Kd are determined by nonlinear curve fitting with KMath in Mathematica 5.0.0, assuming a 1:1:1 stoichiometric, competitive inhibition.

TABLE 1
HuR inhibitors

| Compound | Kd (HuR_{fl}) |
|---|---|
| Example 3 | 0.092 ± 0.022 μM |
| Example 4 | 0.103 ± 0.020 μM |
| Example 5 | 0.104 ± 0.014 μM |
| Example 6 | 0.143 ± 0.027 μM |

Dissociation constants for the compounds are determined based on competition experiments with ARE RNA binding to HuR_{fl}

We claim:

1. Assay for identifying an agent that modulates the interaction of a mRNA with a target protein comprising:
a) providing a labeled mRNA having a length of at least 100 nucleotides, optionally as a homogenous solution, which label is a substance sensitive to changes in the surrounding of the mRNA,
b) contacting a target protein with the mRNA provided in step a) in the absence and in the presence of a candidate compound which is expected to modulate the interaction of said mRNA with said target protein for a sufficient period of time so that a complex between said mRNA and said target protein can be formed,
c) detecting the complex formed in step b),
d) determining whether there is a difference in the amount of complex formed in case a candidate compound was absent or present in step b), and
e) choosing a candidate compound where a difference is determined in step d) as an agent.

2. The assay of claim 1, wherein the label is a fluorescence dye, e.g. Cy3 or Cy5.

3. The assay of claim 1, wherein the complex is detected by measurement of the fluorescence intensity.

4. The assay of claim 1 wherein the target protein is HuR protein.

5. The assay of claim 1 wherein the mRNA is selected from the group consisting of IL-2, IL-1β and TNF-α.

6. The assay of claim 1, wherein the mRNA has a length between 100 and 500 nucleotides, preferably 300 nucleotides.

7. The assay of claim 1 for high throughput screening.

8. A kit comprising:
a labeled mRNA, e.g. fluorescence labeled,
a target protein,
instructions for using such a kit, and
optionally a candidate compound.

9. A compound of formula 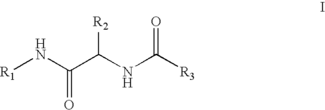
wherein
R₁ is (C₁₋₄)alkyl substituted by unsubstituted or substituted (C₆₋₁₈)aryl or heterocyclyl having 5 or 6 ring meinbers and 1 to 4 heteroatoms selected from the group consisting of N, O and S,
R₂ is (C₁₋₄)alkyl substituted by hydroxyl, carboxyl, amino or unsubstituted or substituted (C₆₋₁₆)aryl, or unsubstituted or substituted (C₆₋₁₈)aryl, and
R₃ is unsubstituted or substituted (C₈₋₁₈)aryl or heterocyclyl having 5 or 6 ring members and 1 to 4 heteroatoms selected from the group consisting of N, O and S.

10. A compound of claim 9, wherein
R₁ is (C₁₋₃)alkyl substituted by unsubstituted or substituted phenyl or heterocyclyl having 5 ring members and N as a heteroatom,
R₂ is (C₁₋₂)alkyl substituted by hydroxyl, carboxy, amino or unsubstituted or substituted phenyl, or unsubstituted or substituted phenyl, and
R₃ is unsubstituted or substituted phenyl or heterocyclyl having 5 or 6 ring members and N as a heteroatom.

11. A compound of formula I of claim 9, wherein
R₁ is methyl substituted by p-methyl-phenyl or n-propyl substituted by 1 pyrrolidin-2-one,
R₂ is methyl substituted by carboxyl, methyl substituted by p-methyl-phenyl, methyl substituted by 1H-indol-3-yl, ethyl substituted by hydroxyl, ethyl substituted by amino or p-methyl-phenyl, and
R₃ is a compound of formula 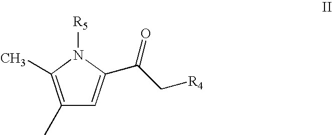
wherein
R₄ is 1-piperidine or 1-(p-aminocarbonyl)-piperidine,
R₅ is methoxyethyl, benzyl or (p-methoxy-phenyl)-ethyl, or
a compound of formula 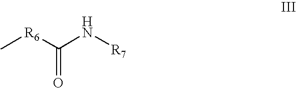
wherein
R₆ is p-phenyl or m-pyridine and
R₇ is methyl substituted by m-methoxy-phenyl or 1-aminocarbonyl-2-hydroxy-propyl.

12. A compound of formula 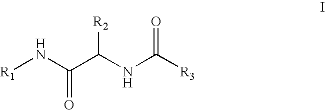
wherein
a) R₁ is n-propyl substituted by 1-pyrrolidin-2-one, R₂ is ethyl substituted by amino and R₃ is a compound of formula 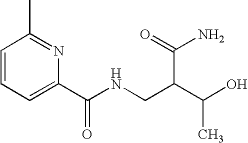
b) R₁ is methyl substituted by p-methyl-phenyl, R₂ is ethyl substituted by amino and R₃ is a compound of formula 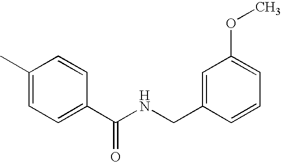
c) R₁ is methyl substituted by p-methyl-phenyl, R₂ is methyl substituted by p-aminomethyl-phenyl and R₃ is a compound as defined in b),
d) R₁ is n-propyl substituted by 1-pyrrolidin-2-one, R₂ is ethyl substituted by hydroxyl and R₃ is a compound of formula 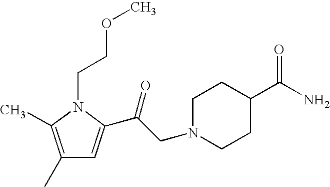
e) R₁ is n propyl substituted by 1-pyrrolidin-2-one, R₂ is methyl substituted by carboxyl and R₃ is a compound of formula 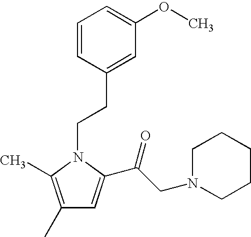
f) R₁ is n-propyl substituted by 1-pyrrolidin-2-one, R₂ is methyl substituted by 1H-indol-3-yl and R₃ is a compound of formula 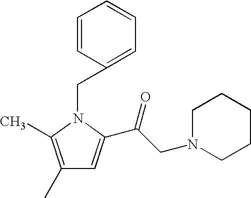
g) R₁ is n-propyl substituted by 1-pyrrolidin-2-one, R₂ is methyl substituted by p-methyl-phenyl and R₃ is a compound as defined in f).

13. A compound of claim 9, in the form of a salt.

14-17. (canceled)

18. A pharmaceutical composition comprising a compound of claim 9 in association with at least one pharmaceutical excipient.

19. A pharmaceutical composition of claim 18, further comprising another pharmaceutically active agent.

20. A method of treatment of a disorder having an etiology associated with the production of a substance selected from the group consisting of cytokine, growth factor, proto-oncogene ora viral protein, preferably selected from the group consisting of IL-1, IL-2, IL-3, IL-4, IL-8, GM-CSF, TNF-α, VEGF, AT-R1, Cox-2, c-fos and c-myc, which treatment comprises administering to a subject in need of such treatment an effective amount of a compound of claim 9.

21. A method of treatment of a disorder having an etiology associated with the production of a substance selected from the group consisting of cytokine, growth factor, proto-oncogene or a viral protein, preferably selected from the group consisting of IL-1, IL-2, IL-3, IL-4, IL-8, GM-CSF, TNF-β, VEGF, AT-R1, Cox-2, c-fos and c-myc, which treatment comprises administering to a subject in need of such treatment an effective amount of a compound of claim 9 wherein the compound is administered in combination with another pharmaceutically active agent, either simultaneously or in sequence.
